(19) United States
(12) Patent Application Publication
(10) Pub. No.: US 2026/0345044 A1
(43) Pub. Date: Nov. 5, 2026

(54) METHOD FOR ASCERTAINING A MAGNETOCARDIOGRAM SIGNAL OF A LIVING BEING

(71) Applicant: Robert Bosch GmbH, Stuttgart (DE)

(72) Inventors: Eckhard Wehrse, Stuttgart (DE); Florian Krist, Stuttgart (DE); Florian Dolde, Stuttgart (DE); Frederik Schaal, Reutlingen (DE); Riccardo Cipolletti, Geislingen An Der Steige (DE)

(21) Appl. No.: 19/099,863

(22) PCT Filed: Aug. 18, 2023
(86) PCT No.: PCT/EP2023/072852

§ 371 Date: Jan. 30, 2025

(30) Foreign Application Priority Data
Sep. 9, 2022 (DE) 10 2022 209 444.1

(51) Int. Cl. A61B 5/243; A61B 5/00

(52) U.S. Cl. CPC A61B 5/243; A61B 5/7203; A61B 5/7267; A61B 5/7278

(57) ABSTRACT

A method for ascertaining a magnetocardiogram signal of a living being. The method includes: sensing a reference magnetic field strength using a sensor unit for measuring magnetic fields; sensing a measured magnetic field strength using the sensor unit when a living being is present in the vicinity of the sensor unit; ascertaining a magnetocardiogram signal of the living based on the reference magnetic field strength and the measured magnetic field strength. A device for ascertaining a magnetocardiogram signal of a living being, a computer program, and a machine-readable storage medium are also described.

### FIELD

The present invention relates to a method for ascertaining a magnetocardiogram signal of a living being, a device for ascertaining a magnetocardiogram signal of a living being, a computer program, and a machine-readable storage medium.

### BACKGROUND INFORMATION

A magnetocardiogram is the recording and representation of the magnetic field of the heart that arises from the electrophysiological activity of the myocardial cells. Usually, this is recorded with highly sensitive magnetic field sensors.

For example, quantum-based magnetic sensors are described in German Patent Application Nos. DE 10 2018 220 234 A1 and DE 10 2018 214 617 A1.

### SUMMARY

An object of the present invention is to provide for efficiently ascertaining a magnetocardiogram signal of a living being.

This object may be achieved by certain features of the present invention. Advantageous example embodiments of the present invention are disclosed herein.

According to a first aspect of the present invention, a method of ascertaining a magnetocardiogram signal of a living being is provided. According to an example embodiment of the present invention, the method comprises the steps of:
sensing a reference magnetic field strength using a sensor unit for measuring magnetic fields,
sensing a measured magnetic field strength using the sensor unit when a living being is present in the vicinity of the sensor unit,
ascertaining a magnetocardiogram signal of the living based on the reference magnetic field strength and the measured magnetic field strength.

According to a second aspect of the present invention, a device for ascertaining a magnetocardiogram signal of a living being is provided. According to an example embodiment of the present invention, the device comprises:
a sensor unit for measuring magnetic fields, wherein the sensor unit is configured to sense a reference magnetic field strength and to sense a measured magnetic field strength in the presence of a living being in the vicinity of the sensor unit, and
an ascertaining means configured to ascertain a magnetocardiogram signal of the living being based on the reference magnetic field strength and the measured magnetic field strength.

According to a third aspect of the present invention, a computer program is provided, which comprises instructions that, when the computer program is executed by a computer, for example by the device according to the second aspect, cause said computer to carry out a method according to the first aspect.

According to a fourth aspect of the present invention, a machine-readable storage medium is provided, on which the computer program according to the third aspect is stored.

The present invention is based on and includes the finding that the above object may achieved by using the sensor unit to sense a magnetic field strength, wherein this magnetic field strength is defined as a reference field strength, i.e., a reference. Subsequently, the measured magnetic field strength is sensed by the sensor unit in the presence of a living being in the vicinity of the sensor unit. The magnetocardiogram signal of the living being is ascertained based on this detected measured magnetic field strength, as well as on the previously ascertained or defined reference.

It is usually the case that the measured magnetic field strength sensed by the sensor unit in the presence of a living being in the vicinity of the sensor unit originates not only from the magnetic field of the heart, but also from further magnetic fields, for example the Earth's magnetic field or, for example, from electrical devices such as household appliances. Such magnetic fields form interfering magnetic fields for the measurement of a cardiac magnetic field. The magnetocardiogram signal may be efficiently ascertained by defining a reference prior to the actual magnetocardiogram measurement, which is considered in the magnetocardiogram measurement which then follows. In particular, interfering magnetic fields may thus be taken into consideration in an efficient manner.

Thus, a long-term magnetocardiogram may be produced in an advantageous manner. Long-term may, for instance, mean multiple hours, for example one night.

Thus, in particular, the technical advantage is that a concept for efficiently ascertaining a magnetocardiogram signal of a living being is provided.

The living being is a human being or an animal, for example.

The abbreviation “MCG” may be used below for the term “magnetocardiogram.”

In one example embodiment of the method of the present invention, it is provided that a background signal representing a magnetic background of the vicinity of the sensor unit is ascertained based on the reference magnetic field strength, wherein the magnetocardiogram signal is ascertained based on the background signal.

This, for example, may produce a technical advantage that the magnetocardiogram signal may be efficiently ascertained. According to this embodiment, therefore, it is provided that the magnetic background of the vicinity of the sensor unit is ascertained based on the reference magnetic field strength and is considered when ascertaining the magnetocardiogram signal.

The magnetic background may result from or be based on the Earth's magnetic field, for example.

In one example embodiment of the method of the present invention, it is provided that an interference signal representing a magnetic interference in the vicinity of the sensor unit is ascertained based on the reference magnetic field strength, wherein the magnetocardiogram signal is ascertained based on the interference signal.

This, for example, may produce a technical advantage that the magnetocardiogram signal may be efficiently ascertained. According to this example embodiment, it is thus provided that a magnetic interference in the vicinity of the sensor unit is ascertained based on the reference magnetic field strength, which is taken into consideration in ascertaining the magnetocardiogram signal. For example, a magnetic interference may result from operating an electrical device in the vicinity of the sensor unit. Such an electrical device, for example, is an electrical household appliance or, for example, is a drive motor of an electrical roller shutter.

If, for example, the sensor unit is in a vehicle, such as an automobile or an aircraft, such magnetic interference may result from operation of the vehicle.

For example, such magnetic interference may result from an electrically powered rail vehicle traveling in the vicinity of the sensor unit. The current draw of such a rail vehicle may create magnetic fields that may interfere with the MCG measurement.

In one example embodiment of the method of the present invention, it is provided that the signal to be ascertained based on the reference magnetic field strength, i.e., the background signal and/or the interference signal, is ascertained using a pattern detection method, in particular a machine-learning method.

This, for example, may produce a technical advantage that the background signal or interference signal may be efficiently ascertained.

In one example embodiment of the method of the present invention, it is provided that the signal to be ascertained based on the reference magnetic field strength, i.e., the background signal and/or the interference signal, is filtered out of a measured signal representing the sensed measured magnetic field strength to obtain a filtered measurement signal, wherein the magnetocardiogram signal is ascertained based on the filtered measurement signal.

This, for example, may produce a technical advantage that the magnetocardiogram signal may be efficiently ascertained. According to this example embodiment of the present invention, it is therefore provided that the magnetic background and/or magnetic interference is filtered out of the sensed measured magnetic field strength.

In one example embodiment of the method of the present invention, it is provided that at least one time interval is ascertained, in particular estimated, within which the magnetic interference occurs and/or will occur, wherein only a measured magnetic field strength sensed outside the at least one time interval is used to ascertain the magnetocardiogram signal.

This, for example, may produce a technical advantage that the magnetocardiogram signal may be efficiently ascertained. According to this embodiment, it is thus provided that detected magnetic interferences are not taken into account when ascertaining the magnetocardiogram signal. On the one hand, this allows measurements, i.e., the magnetocardiogram measurement, to be carried out only outside of the time interval. On the other hand, additionally or alternatively, an MCG measurement carried out within the time interval cannot be considered when ascertaining the magnetocardiogram signal.

In one example embodiment of the method of the present invention, it is provided that at least one vital sign of the living being is ascertained based on the magnetocardiogram signal.

This, for example, may bring about a technical advantage that the at least one vital sign of the living being may be ascertained efficiently.

In one example embodiment of the method of the present invention, it is provided that the at least one vital sign is an element selected from the following group of vital signs: heart rate, heart rate variability, duration and/or amplitude of an ECG equivalent signal change, for example P-wave, QRS complex, T-wave, and corresponding combinations.

This, for example, may bring about a technical advantage that particularly suitable vital signs may be ascertained efficiently.

In one example embodiment of the method of the present invention, it is provided that the measuring range comprises a section of an object, which is an element selected from the following group of objects: mattress, pillow, bed, chair, couch, armchair, head support, seat, vehicle seat, aircraft seat, airplane seat, automotive seat.

This, for example, may bring about a technical advantage that particularly suitable measuring ranges may be provided.

In one example embodiment of the device of the present invention, it is provided that the measuring range comprises a section of an object, which is an element selected from the following group of objects: mattress, pillow, bed, chair, couch, armchair, head support, seat, vehicle seat, aircraft seat, airplane seat, automotive seat.

This, for example, may bring about a technical advantage that particularly suitable measuring ranges may be used.

Technical functionalities of the device of the present invention result analogously from corresponding technical functionalities of the method of the present invention and vice versa. This means that device features result in particular from corresponding method features and vice versa.

For example, it is specified that the device of the present invention is configured to carry out all steps of the method according to the first aspect of the present invention.

For example, it is provided that the method according to the first aspect of the present invention is executed or performed by means of the device according to the second aspect of the present invention.

According to one example embodiment of the present invention, the method is a computer-implemented method.

The phrase “at least one” means “one or more.”

The example embodiments described here may be combined with one another in any manner, even if this is not explicitly described.

The reference magnetic field strength is sensed by the sensor unit, for example in the absence and/or in the presence of a living being in the vicinity of the sensor unit.

For example, the measured magnetic field strength sensed in the presence of a living being in the vicinity of the sensor unit may be used as the reference magnetic field strength for a later MCG measurement. This means that an MCG measurement may also be used as a reference for MCG measurements taken at a later time. Thus, in an advantageous manner, magnetic interferences that do not occur regularly over time or that did not occur while, for example, the reference magnetic field strength was being sensed, may still be detected and considered for an MCG measurement.

An MCG measurement comprises using the sensor unit to sense the measured magnetic field strength in the presence of a living being in the vicinity of the sensor unit and ascertaining the magnetocardiogram signal. The output or result of the MCG measurement is the magnetocardiogram signal.

For example, multiple magnetocardiogram signals may be ascertained. Statements made in connection with a magnetocardiogram signal apply analogously to multiple magnetocardiogram signals and vice versa.

In one example embodiment of the method of the present invention, a distribution of interference signals over time and/or a spectral distribution of the interference signals are ascertained, wherein the magnetocardiogram signal is ascertained based on the respective distribution.

This, for example, produces the technical advantage that the magnetocardiogram signal may be efficiently ascertained.

The present invention is explained in more detail in the following with reference to preferred embodiment examples.

### BRIEF DESCRIPTION OF THE DRAWINGS

FIG. 1 shows a flowchart of a method for ascertaining a magnetocardiogram signal of a living being, according to an example embodiment of the present invention.

FIG. 2 shows a device for ascertaining a magnetocardiogram signal of a living being, according to an example embodiment of the present invention.

FIG. 3 a machine-readable storage medium, according to an example embodiment of the present invention.

FIG. 4 shows multiple interfering signals measured over several consecutive nights, according to an example embodiment of the present invention.

### DETAILED DESCRIPTION OF EXAMPLE EMBODIMENTS

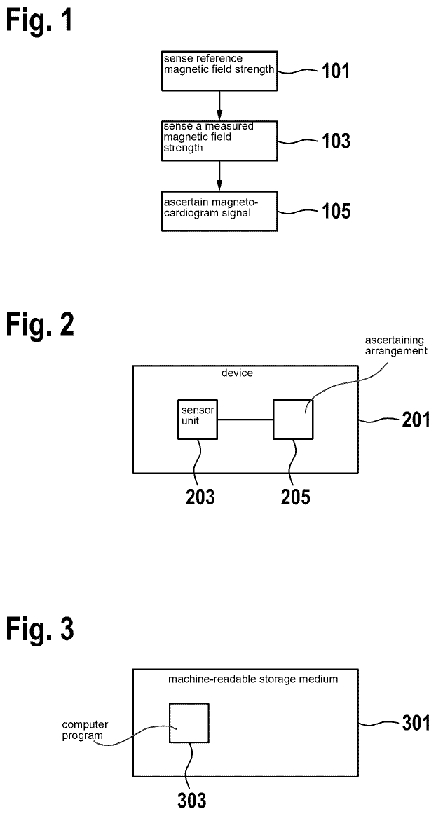

FIG. 1 shows a flowchart of a method for ascertaining a magnetocardiogram signal of a living being, comprising the steps of:
sensing 101 a reference magnetic field strength using a sensor unit for measuring magnetic fields,
sensing 103 a measured magnetic field strength using the sensor unit in the presence of a living being in the vicinity of the sensor unit,
ascertaining 105 a magnetocardiogram signal of the living being based on the reference magnetic field strength and the measured magnetic field strength.

FIG. 2 shows a device 201 for ascertaining a magnetocardiogram signal of a living being, comprising:
a sensor unit 203 for measuring magnetic fields, wherein the sensor unit 203 is configured to sense a reference magnetic field strength and sense a measured magnetic field strength in the presence of a living being in the vicinity of the sensor unit 203, and
an ascertaining means 205 configured to ascertain a magnetocardiogram signal of the living being based on the reference magnetic field strength and the measured magnetic field strength.

FIG. 3 shows a machine-readable storage medium 301, on which a computer program 303 is stored. The computer program 303 comprises instructions that, when the computer program 303 is executed by a computer, cause it to perform a method of ascertaining a magnetocardiogram signal of a living being.

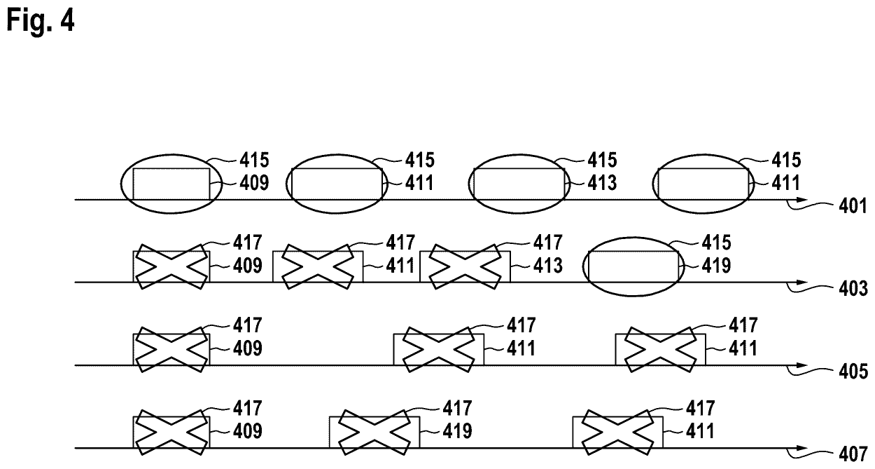

FIG. 4 shows a first time axis 401, a second time axis 403, a third time axis 405, and a fourth time axis 407. The four time axes denote four consecutive nights, which may also be subsequently referred to as night 1, night 2, night 3, and night 4.

A device for ascertaining a magnetocardiogram signal is provided. For example, the sensor unit is integrated in a mattress of a bed. For example, the ascertaining device may be provided outside of the mattress or may also be located within the mattress.

In order to sense a reference field strength, it is provided that the sensor unit takes a measurement of magnetic fields, i.e., a magnetic field strength, during night 1 without a living being being in the vicinity of the sensor unit, i.e., in the absence of a living being in the vicinity of the sensor unit.

This means that magnetic fields may be recorded or captured by means of the device during night 1. Such magnetic fields represent magnetic interference fields for later MCG measurement in the nights night 2 to night 4.

By way of example, three magnetic interference fields were measured during night 1: a first interference signal 409, a second interference signal 411, and a third interference signal 413. The second interference signal 411 occurred twice during night 1, as is shown by the first time axis 401.

In addition, an oval with the reference number 415 was drawn around the interference signals 409, 411, 413 to indicate that these interference fields or interference signals have not yet been filtered out of an MCG measurement signal.

However, in the subsequent nights night 2 to night 4, these interference signals may be filtered out of the measurement field strengths that are then measured. This is symbolically indicated by an X with the reference number 417. This means that in the subsequent nights night 2 to night 4, a living being, for example a human being, is on the mattress. Further, it is provided that during the nights night 2 to night 4, the sensor unit will detect or measure magnetic field strengths, the measured magnetic field strengths. Measurement signals corresponding to this measurement may then serve as the basis for a magnetocardiogram signal of the living being. It is provided that the interference signals ascertained during night 1, i.e., the reference night, are filtered out of the measurement signals that were sensed in the next night 2 to night 4.

As an example, it is shown that a fourth magnetic interference field 419 was sensed in night 2, which may also be filtered out in the following nights, night 3 and night 4.

Detection of such interference fields may be identified using, for example, a machine learning algorithm or artificial intelligence. For example, a pattern detection method may be used to detect patterns in the reference magnetic field strengths, which may then be filtered out of the measurement signal which is then present in the nights night 2 to night 4.

In summary, the present invention described here is based on, for example, a reference measurement and a pattern detection based on the reference measurement, so that detected patterns may be considered when ascertaining magnetocardiogram signals of a living being. For example, it is provided that a background signal and/or an interference signal will also be ascertained based on the measured magnetic field strength, which may then be used for further MCG measurements to filter corresponding magnetic background and/or corresponding magnetic interference out of the measurement signal. This means that it is also possible to ascertain a magnetic background and/or to ascertain a magnetic interference analogously to the reference magnetic field strength for the measured magnetic field strength. The corresponding statements apply analogously in this respect. Thus, the measured magnetic field strength may also be evaluated using a pattern detection method, in particular a machine-learning method, to ascertain the background signal and/or the interference signal. Thus, these ascertained signals may be used for subsequent MCG measurements to ascertain the corresponding magnetocardiogram signal of the living being based on these signals.

For example, efficient noise suppression may be advantageously achieved. Thus, it may further be advantageously provided to give feedback as to whether the device is working without restrictions or only to a limited extent and, for example, how long this state is expected to continue. In particular, a distribution of interference signals over time and/or a spectral distribution of the interference signals is taken into consideration.

To identify a background signal or an interference signal, respectively, for example, the method provides for it to be measured. For this purpose, for example, an initial reference measurement is carried out, i.e., a measurement of reference magnetic field strengths, for example, for one night without a living being present in the vicinity of the sensor unit. The vicinity of the sensor unit may also generally be referred to as a measuring range.

Then, a magnetocardiogram measurement may be performed on a living being that is in the measuring range during the subsequent nights. This means that when the living being is in the vicinity of the sensor unit, a measured magnetic field strength is sensed by means of the sensor unit.

The fact that the reference measurement may be carried out for one night is to be understood by way of example. Shorter periods, for example a few minutes, may also be provided. For example, a few minutes is sufficient during self-calibration of the sensor unit and/or the device. This means, for example, that an embodiment of the method is performed during self-calibration of the sensor unit and/or the device. This means in particular that method steps are carried out, for example during self-calibration of the sensor unit and/or the device. A few minutes are also sufficient, for example, if the method is applied or is to be applied in a vehicle, for example a motor vehicle, or in a hospital.

For example, it is generally provided that the reference magnetic field strength be subtracted from the measured magnetic field strength. This is done in particular synchronous with the time of day, and in general with the time. For example, the reference signal is subtracted from the measurement signal, wherein the magnetocardiogram signal is ascertained, for example, based on the corresponding subtracted measurement signal. For example, it is provided that at times or time intervals at which interference signals and/or background signals occur according to the reference magnetic field strength, corresponding filters are provided that filter out these interference signals and/or background signals from the measurement signals. In this way, for example, interference signals that always occur at the same time may be efficiently filtered out. For example, a magnetic interference caused by a drive motor of an electric roller shutter may be efficiently filtered out. For example, based on the reference magnetic field strength, it may be ascertained that the shutters move up or down at a particular time, such that an interference signal may be expected to occur at these times, such that at these times this interference signal may be filtered out correspondingly in the measurement signal.

For example, patterns in the reference magnetic field strengths may be detected. Such patterns may also be learned from the timing of the interference signal. This means that if it is known that interference always occurs at the same time, then the course of the reference magnetic field strength at these times is defined as the pattern to be filtered out. An example is when an electrically driven rail vehicle travels in the vicinity of the sensor unit at approximately the same or similar times and thus interferes with the MCG measurement by drawing current for the drive motor. Thus, the filters may be adjusted for data analysis in particular in an efficient manner for specific times of day or night.

Classic pattern detection methods may be used and/or machine learning may be used for this purpose. For example, characteristic noise patterns may be identified in the reference data, i.e., the sensed reference magnetic field strength, for example, a passing of a rail vehicle, such as a tram, or the operation of an elevator. The detected noise patterns may then be removed from the measured signal, i.e., the measurement signal, for example, for noise suppression, or the corresponding filter criteria may be learned.

Further patterns may thus be identified by continuous analysis of the measurement data, i.e., the measured magnetic field strength, and data quality may be advantageously improved.

By identifying points in time or patterns with particularly high noise, for example, it is possible to identify periods of reduced functionality with respect to MCG measurement. This makes it possible for these time periods to be excluded from further analyses or allows feedback to be provided on the non-functionality of the device with regard to the MCG measurement. It is in particular advantageous that a prediction about the expected duration and the degree of interference may be provided here by identifying patterns. Data weighted according to the patterns described in this way may then be managed in, for example, a database, which is, for example, online, and may be utilized in a data evaluation and with respect to an associated function, for example, an alert that functionality is restricted.

1-12. (canceled)

13. A method for ascertaining a magnetocardiogram signal of a living being, comprising the following steps:
sensing a reference magnetic field strength using a sensor unit for measuring magnetic fields;
sensing a measured magnetic field strength using the sensor unit when a living being is present in a vicinity of the sensor unit; and
ascertaining a magnetocardiogram signal of the living based on the reference magnetic field strength and the measured magnetic field strength.

14. The method according to claim 13, wherein a background signal representing a magnetic background of the vicinity of the sensor unit is ascertained based on the reference magnetic field strength, wherein the magnetocardiogram signal is ascertained based on the background signal.

15. The method according to claim 13, wherein an interference signal representing a magnetic interference in the vicinity of the sensor unit is ascertained based on the reference magnetic field strength, wherein the magnetocardiogram signal is ascertained based on the interference signal.

16. The method according to claim 14, wherein the background signal to be ascertained based on the reference magnetic field strength, is ascertained using a pattern detection method including a machine-learning method.

17. The method according to claim 14, wherein the background signal to be ascertained based on the reference magnetic field strength is filtered out of a measurement signal representing the sensed measured magnetic field strength to obtain a filtered measurement signal, wherein the magnetocardiogram signal is ascertained based on the filtered measurement signal.

18. The method according to claim 15, wherein at least one time interval is estimated within which the magnetic interference occurs and/or will occur, wherein only a measured magnetic field strength sensed outside of the at least one time interval is used to ascertain the magnetocardiogram signal.

19. The method according to claim 13, wherein at least one vital sign of the living being is ascertained based on the magnetocardiogram signal.

20. The method according to claim 19, wherein the at least one vital sign is at least one of the following elements: heart rate, heart rate variability, duration and/or amplitude of an ECG equivalent signal change, P-wave, QRS complex, T-wave.

21. A device configured to ascertain a magnetocardiogram signal of a living being, comprising:
a sensor unit configured to measure magnetic fields, wherein the sensor unit is configured to sense a reference magnetic field strength, and to sense a measured magnetic field strength in a presence of a living being in the vicinity of the sensor unit; and
an ascertaining arrangement configured to ascertain a magnetocardiogram signal of the living being based on the reference magnetic field strength and the measured magnetic field strength.

22. The device according to claim 21, wherein the measuring range includes a section of an object, which is an element selected from the following group of objects: mattress, pillow, bed, chair, couch, armchair, head support, seat, vehicle seat, aircraft seat, airplane seat, automotive seat.

23. A non-transitory machine-readable storage medium on which is stored a computer program for ascertaining a magnetocardiogram signal of a living being, the computer program, when executed by a computer, causing the computer to perform the following steps:
sensing a reference magnetic field strength using a sensor unit for measuring magnetic fields;
sensing a measured magnetic field strength using the sensor unit when a living being is present in a vicinity of the sensor unit; and
ascertaining a magnetocardiogram signal of the living based on the reference magnetic field strength and the measured magnetic field strength.
